(19) United States
(12) Patent Application Publication
(10) Pub. No.: US 2013/0132111 A1
(43) Pub. Date: May 23, 2013

(54) Pharmaceutical Business Method for High Volume Low Risk Sales

(71) Applicant: David Brent Morgan, Clinton, UT (US)

(72) Inventor: David Brent Morgan, Clinton, UT (US)

(21) Appl. No.: 13/674,028

(22) Filed: Nov. 10, 2012

Related U.S. Application Data
(60) Provisional application No. 61/563,388, filed on Nov. 23, 2011.

(51) Int. Cl. G06Q 50/22

(52) U.S. Cl. 705/2

(57) ABSTRACT

A business method for pharmaceutical sales in which a single or multiple additional parties are introduced into the transaction such that a push market strategy results for the sales of pharmaceutical which are currently sold under patent licensing. The method promotes the widespread adoption of patented pharmaceuticals while reducing risk and increasing sales volume through guaranteed long term bulk purchase agreements. The resulting method lowers initial consumer prices of pharmaceuticals during the onset of early phase production while leveling prices toward the final phase of patent life.

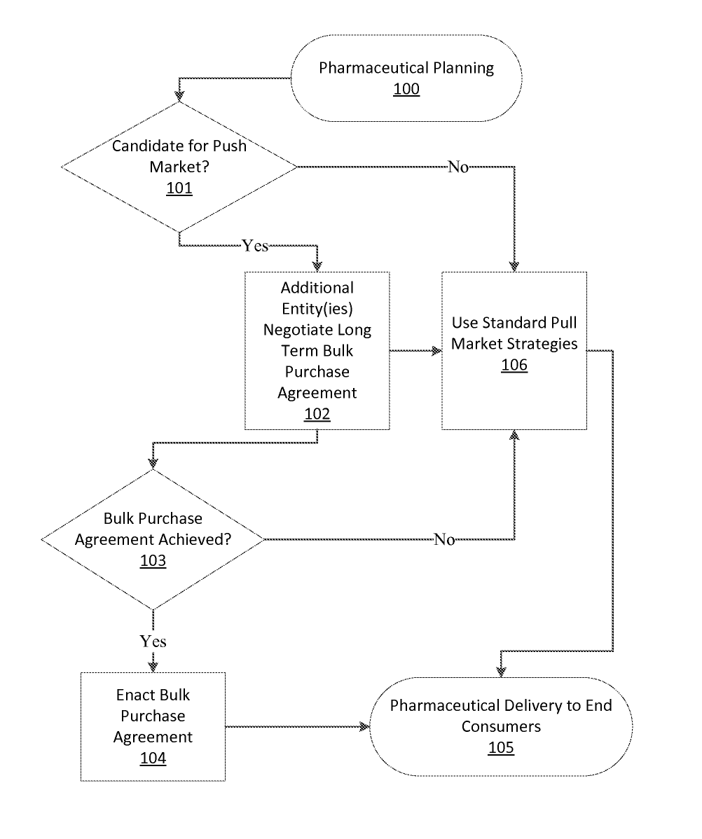

This application claims the priority of U.S. patent application Ser. No. 61/563,388 filed on Nov. 23, 2011 entitled Pharmaceutical Business Method for High Volume Low Risk Sales.

### FIELD OF THE INVENTION

The present invention pertains to the field of pharmaceutical sales. More specifically, the invention relates to a pharmaceutical business method for high volume and low risk sales that allows a push market strategy versus the traditional pull market strategy.

### BACKGROUND

For decades the pharmaceutical industry has relied upon a pull market wherein the consumer is the market driver. Initial research and development costs can be staggering for the pharmaceutical industry and thus must be recaptured in a fairly short timeframe with the initial deployment of a new pharmaceutical. This pull market strategy has resulted in low initial sales coupled with staggering expenses in marketing thus increasing the cost of newly released pharmaceuticals for consumers. While the research and development costs as well as the marketing costs are eventually recaptured as the pharmaceutical matures, the pharmaceutical patent holder eventually faces market pressure from generic manufacturers as the patent expires. The resulting effect is a cost curve such that consumers of small means simply cannot afford a pharmaceutical until the end of a patent cycle when a generic equivalent and market competition is introduced. The resulting effect is a pharmaceutical patent holder misses a potentially large market of consumers.

The dilemma of pharmaceuticals missing a potentially large market in such cases as developing nations in great need of patented vaccines results in many cases of preventable spread of disease and even death until market prices reach such a level that the vaccine becomes affordable. The inability of pharmaceutical patent holders and consumers to close the gap between early adoption and wide market penetration has become problematic.

### SUMMARY OF THE INVENTION

In accordance with the present invention, a pharmaceutical business method for high volume low risk sales is provided that overcome the known problems with the gap between early adoption and wide market penetration.

In particular, a method for high volume low risk pharmaceutical sales is provided that allows a push market strategy versus the traditional pull market strategy.

In accordance with an exemplary embodiment of the present invention, a pharmaceutical business method for high volume low risk sales is provided. The method includes a decision making process for determining market strategy and negotiating purchase agreements, and delivery of pharmaceuticals to end consumers.

The present invention provides many important advantages. One important advantage is the sales and delivery of pharmaceuticals to potential markets heretofore unrealized in the patented pharmaceutical market. The present invention thus allows a sustainable business model which permits developing nations access to most vital pharmaceutical vaccines for large populations who would otherwise not have feasible access to such vaccines; resulting in the diminishing spread of disease and even untimely patient death among developing nations.

Those skilled in the art will further appreciate the advantages and superior features of the invention together with important aspects thereof on reading the detailed description that follows in conjunction with the drawings.

### BRIEF DESCRIPTION OF THE DRAWINGS

The drawing FIGURE is a flow chart of a pharmaceutical business method for high volume low risk sales in accordance with an exemplary embodiment of the present invention.

### DETAILED DESCRIPTION OF PREFERRED EMBODIMENTS

In the description which follows, like parts are marked throughout the specification and drawings with the same reference numerals, respectively. The drawing figure may not be to scale and certain components can be shown in generalized or schematic form and identified by commercial designation in the interest of clarity and conciseness.

The drawing figure is a flow chart of a pharmaceutical business method for high volume low risk sales in accordance with an exemplary embodiment of the present invention. During pharmaceutical planning 100 or any time thereafter, a decision could be made if the pharmaceutical is a candidate for utilizing a push market strategy 101. To achieve a push market strategy 101 one or more additional entities or parties must become involved. These entities or parties negotiate a long term bulk purchase agreement 102 with the pharmaceutical patent holder. A successful agreement 103 would include elements such that the additional entities or parties are able to secure a reasonable price and guarantee a long term purchase contract such that the pharmaceutical patent holder could recoup necessary costs and expected profit margin over the life of the patent. Once the agreement is enacted 104 and the pharmaceutical is available, delivery of the pharmaceutical could begin to the end consumers 105. If the pharmaceutical is not a good candidate for a push market strategy 101, the pull market strategy 106 is permitted as part of the bulk purchase agreement 102, or the bulk purchase agreement fails 103; a standard pull market strategy 106 could be utilized for delivery of the pharmaceutical to end consumers.

The expected results are a pharmaceutical business method for high volume low risk sales. The pharmaceutical patent holder benefits from a bulk volume of sales in a low risk format due to the nature of the bulk purchase agreement 102. Furthermore, the bulk purchase agreement 102 could also include provisions attractive to the pharmaceutical patent holder to further lower risk such as a reduction in liability or guaranteed liability limit.

The expected results for the end consumers, that are the beneficiaries of the bulk purchase agreement 102, are lower and affordable prices for newly developed pharmaceuticals. A successful and enacted bulk purchase agreement 104, such as in the case of a vaccine in a developing nation, will yield health benefits that would otherwise go unrealized preventing the spread of disease and even untimely death of patients.

The term pharmaceutical patent hold is defined herein as any party or entity that has the intent to manufacture, produce, market, or sell patented pharmaceuticals. The terms entity or party are defined herein as any government, business, person, association, institute, organization, non-profit, or any other organized collection of people. The terms long term bulk purchase agreement and bulk purchase agreement are defined as any treaty, accord, arrangement, agreement, contract, bond, pledge, promise, pact, negotiation, or accord, or anything similar whether it be verbal or written for the purchase, distribution, or sales of any patented pharmaceutical over any period of time.

Although preferred and exemplary embodiments of a pharmaceutical business method for high volume and low risk sales have been described in detail herein, those skilled in the art will also recognize that various substitutions and modification can be made to the methods without departing from the scope and spirit of the appended claims.

What is claimed is:

1. A method for high volume and low risk pharmaceutical sales; wherein a push market strategy, versus the standard pull market strategy, is achieved.

2. The method of claim 1 wherein one or more additional parties or entities negotiate a pull market strategy.

3. The method of claim 2 wherein a bulk purchase agreement is negotiated by said one or more additional parties or entities.

4. The method of claim 3 wherein the said bulk purchase agreement is enacted.
